(19) United States
(12) Patent Application Publication
(10) Pub. No.: US 2018/0360364 A1
(43) Pub. Date: Dec. 20, 2018

(54) Needle Guard

(71) Applicant: Chris WHELAN, Leichhardt, NSW (AU)

(72) Inventor: Chris WHELAN, Leichhardt, NSW (AU)

(21) Appl. No.: 15/739,171

(22) PCT Filed: Jun. 27, 2016
(86) PCT No.: PCT/AU2016/000232

§ 371 Date: Dec. 22, 2017

Related U.S. Application Data
(60) Provisional application No. 62/185,092, filed on Jun. 26, 2015.
(60) Provisional application No. 62/275,326, filed on Jan. 6, 2016.

(51) Int. Cl. A61B 5/15; A61M 5/158; A61M 5/50; A61B 5/153

(52) U.S. Cl. CPC A61B 5/150641; A61B 5/15003; A61B 5/150916; A61M 25/06; A61M 5/50; A61B 5/153; A61M 5/158

(57) ABSTRACT

A needle guard (10) has an elongate concave channel member (22) having a longitudinally extending opening between two longitudinally extending sides (30) and defining part of a passageway (36) having a forward end (40) and a rear end (64). A tongue member (24), in an open position, extends longitudinally adjacent the opening and defines at least one slot (34) between itself and at least one of the sides (30) adapted to receive at least one wing of a winged needle block. The tongue member (24) is movable to a closed position to lie within the channel member (22) to close, block or substantially close or block the passageway (36).

### FIELD OF INVENTION

This invention relates to needle guards and more particularly to guards into which a winged needle assembly having a needle at an end is withdrawn into.

### BACKGROUND

Needle guards are protective devices into which a needle of a needle assembly can be withdrawn. Once fully withdrawn the needle is locked within the guard and cannot be accidentally extended, thus preventing accidental needle stick injury once it is within the guard.

Winged needle assemblies have a needle block with one or two flexible wings that allow the user to more easily manipulate the needle assembly.

### SUMMARY OF THE INVENTION

The present invention aims to provide a novel needle guard for use with winged needle assemblies. At least some preferred embodiments aim to provide easier withdrawal into the guard, substantial sealing against liquid leakage and visual and audible feedback to the user to have confidence the withdrawn needle assembly is secured correctly.

The present invention provides a needle guard for use with a needle assembly having a needle mounted on a needle block, the guard comprising:
an elongate concave channel member having a longitudinally extending opening between two longitudinally extending side edges, the concave channel member defining at least part of a passageway for receiving at least part of a needle assembly;
an elongate tongue member, in an open position, extending longitudinally adjacent the opening and defining at least one slot between itself and at least one of the side edges, the at least one slot adapted to receive at least one wing of a winged needle block;
at least a portion of the tongue member movable to a closed position to lie within the channel member to close, block or substantially close or block the passageway.

Preferably reception of at least one wing in the at least one slot does not move said tongue member relative to the channel member.

Preferably in the open position the tongue member lies outside the channel member. However, at least part of the tongue member may lie within the channel member.

In a preferred embodiment, when viewed side on, there is gap between the at least one side edge and the tongue member. This allows a winged needle block to be drawn into the Guard with the wings passing along the slots with no or insignificant deflection of the wings.

However, at least part of the tongue member may lie within the channel member such that, when viewed side on, there is no gap between the at least one side edge and the tongue member. The slot so defined allows a winged needle block to be drawn into the guard with the wings passing along the slots but requiring some deflection.

Preferably there is a slot defined between the tongue member and both side edges.

Preferably in the closed position the tongue member closes at least part of the at least one slot.

The needle guard preferably has at least one locking mechanism to retain the tongue in the closed position. In a preferred embodiment the locking mechanism comprises at least one protrusion on one of the tongue or the channel member and at least one recess or opening on the other for receiving the at least one protrusion. In a preferred embodiment the tongue has protrusions that engage in recesses or openings in the channel member.

Preferably in the closed position the tongue member is located within the channel member. However, in the closed position the tongue member may bear against the side edges to close the slot and have a closure portion that extends into the channel member to close the passageway.

The concave channel member may comprise a top wall and two opposed first side walls.

The tongue member may comprise a lower wall. The tongue member may have two opposed second side walls.

Preferably the separation of the first side walls is more than the width of the tongue, excluding any locking protrusions or recesses on the side walls and tongue.

The tongue preferably includes sealing structure to seal the front end of the passageway against leakage of liquid from the passageway to the environment via the front end.

In a preferred embodiment, in the open position, the at least one slot communicates with at least one wing-receiving recess, for receiving at least one wing of a winged needle block.

At least part of the at least one wing-receiving recess may be defined by the tongue member or the channel member or both the tongue member and the channel member.

Preferably, in the closed position, the wing-receiving recess is closed.

The wing-receiving portion may have at least one wing-receiving slot, for receiving a front portion of a needle block wing. The at least one wing-receiving slot may be formed in the tongue member or the channel member. The at least one wing-receiving slot may be rearward facing.

The guard preferably includes at least one guide member or surface to align the needle assembly to a first position when retracted into the needle guard.

The at least one guide member or surface may be fixed relative to one or both of the tongue member and the channel member or it may be movable relative to one or both of the tongue member and the channel member.

The at least one guide member or surface may include a slot or movable arm. The at least one guide member or surface may comprise part of the wing-receiving portion or wing-receiving slot.

The guard preferably includes at least one biasing member to bias a needle assembly to a first position when retracted into the needle guard. The at least one guide member or surface may include at least one biasing member. The at least one biasing member may include at least one guide member or surface.

The at least one biasing member may comprise a flexible arm located on or extending from either or both of the tongue member and the channel member.

The at least one biasing member may bias the needle assembly, when in the retracted position, toward the tongue or the channel member.

The at least one biasing member may bias the needle assembly, when in the retracted position, toward the tongue or the channel member so that a portion of the cannula at or near the free end of the cannula is close to or engages the surface of the respective tongue or the channel member.

### BRIEF DESCRIPTION OF THE DRAWINGS

FIG. 1 is a perspective view from above of a needle guard according to a first embodiment of the invention;

FIG. 2 is a perspective view from below of the needle guard of FIG. 1;

FIG. 3 is a side view of the needle guard of FIG. 1 in an open position;

FIG. 4 is a cross sectional view the needle guard of taken along line AA of FIG. 3;

FIG. 5 is a front view of the needle guard of FIG. 1 in an open position;

FIG. 6 is a perspective longitudinal cross sectional view the needle guard taken along line BB of FIG. 5;

FIG. 7 is a longitudinal cross sectional view the needle guard of FIG. 1 in the open position;

FIG. 8 is a longitudinal cross sectional view the needle guard of FIG. 1 in the closed position;

FIG. 9 is a side view of the needle guard of FIG. 1 mounted on a needle assembly when in an open position;

FIG. 10 is a plan view of the needle guard assembly of FIG. 9;

FIG. 11 is a longitudinal cross sectional view the needle guard assembly taken along line AA of FIG. 10;

FIG. 12 is side view the needle guard assembly of FIG. 9 but with a retracted needle assembly;

FIG. 13 is a plan view of the needle guard assembly of FIG. 12;

FIG. 14 is a longitudinal cross sectional view the needle guard assembly taken along line AA of FIG. 13;

FIG. 15 is a longitudinal cross sectional view the needle guard assembly taken along line AA of FIG. 13 but with the needle guard locked in a closed position;

FIG. 16 is a detail view of the end of the needle guard circled B in FIG. 15.

FIG. 17 is a perspective view from above of a needle guard according to a second embodiment of the invention mounted on a needle assembly;

FIG. 18 is a side view of the needle guard of FIG. 17 in an open position;

FIG. 19 is a front view of the needle guard of FIG. 17 in an open position;

FIG. 20 is a plan view of the needle guard assembly of FIG. 17;

FIG. 21 is a side view of the needle guard assembly of FIG. 17;

FIG. 22 is a longitudinal cross sectional view of the needle guard assembly taken along line AA of FIG. 20;

FIG. 23 is side view the needle guard assembly of FIG. 17 but with the needle assembly retracted;

FIG. 24 is a longitudinal cross sectional view the needle guard assembly of FIG. 23;

FIG. 25 is a perspective view the needle guard assembly of FIG. 23;

FIG. 26 is a side view the needle guard of FIG. 20 in the closed position;

FIG. 27 is a longitudinal cross sectional view the needle guard assembly of FIG. 26;

FIG. 28 is a cross sectional view of the needle guard assembly taken along line BB of FIG. 26;

FIG. 29 is a perspective view from above of a needle guard according to a second embodiment of the invention mounted on a needle assembly;

FIG. 30 is a side view of the needle guard of FIG. 17 in an open position;

FIG. 31 is a perspective view the needle guard assembly of FIG. 29 but with the needle assembly retracted;

FIG. 32 is a side view the needle guard assembly of FIG. 29 but with the needle assembly retracted;

FIG. 33 is a side view the needle guard assembly of FIG. 29 but with the needle assembly retracted and the needle guard closed.

### DETAILED DESCRIPTION OF PREFERRED AND OTHER EMBODIMENTS

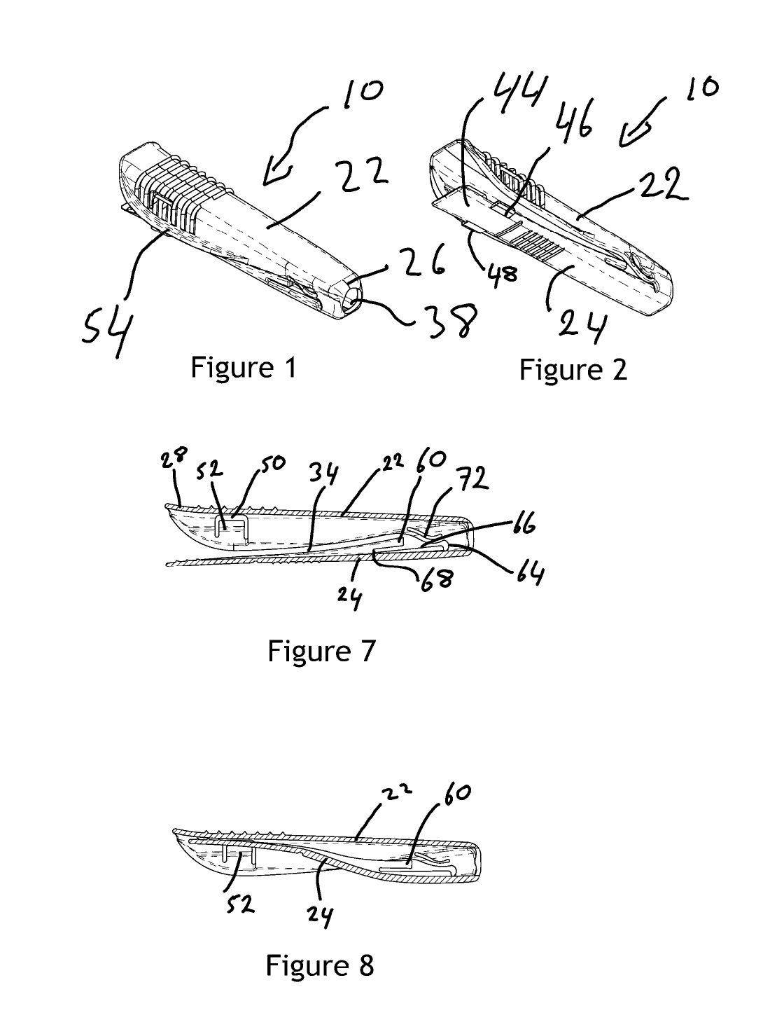

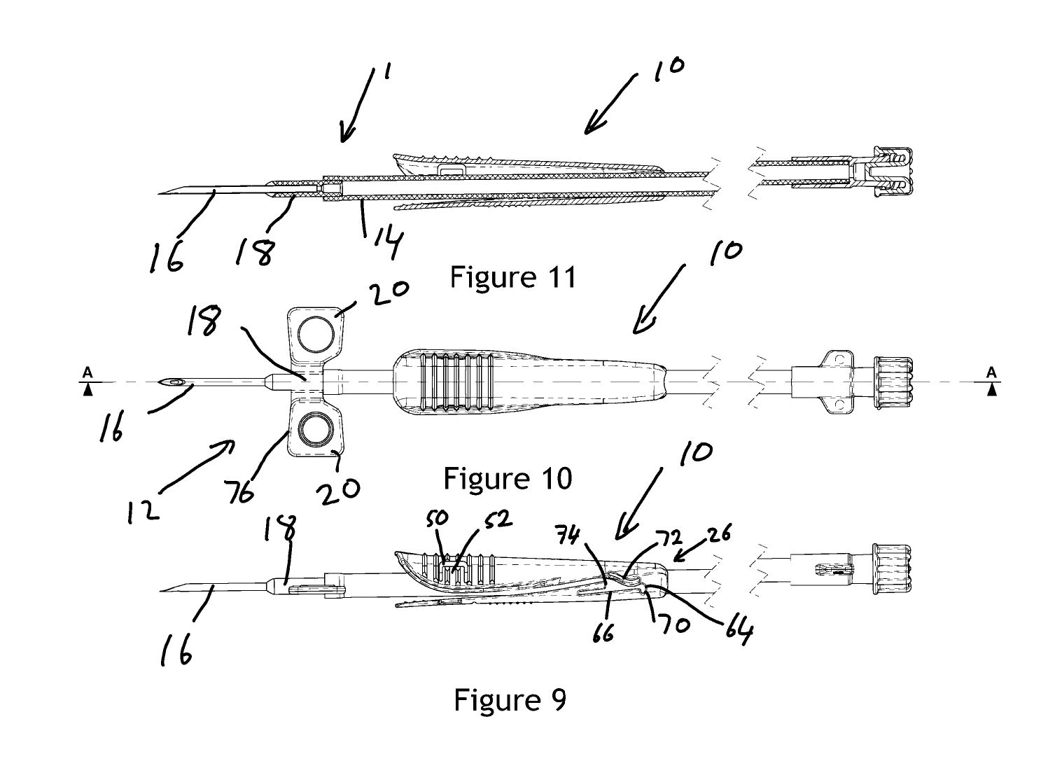

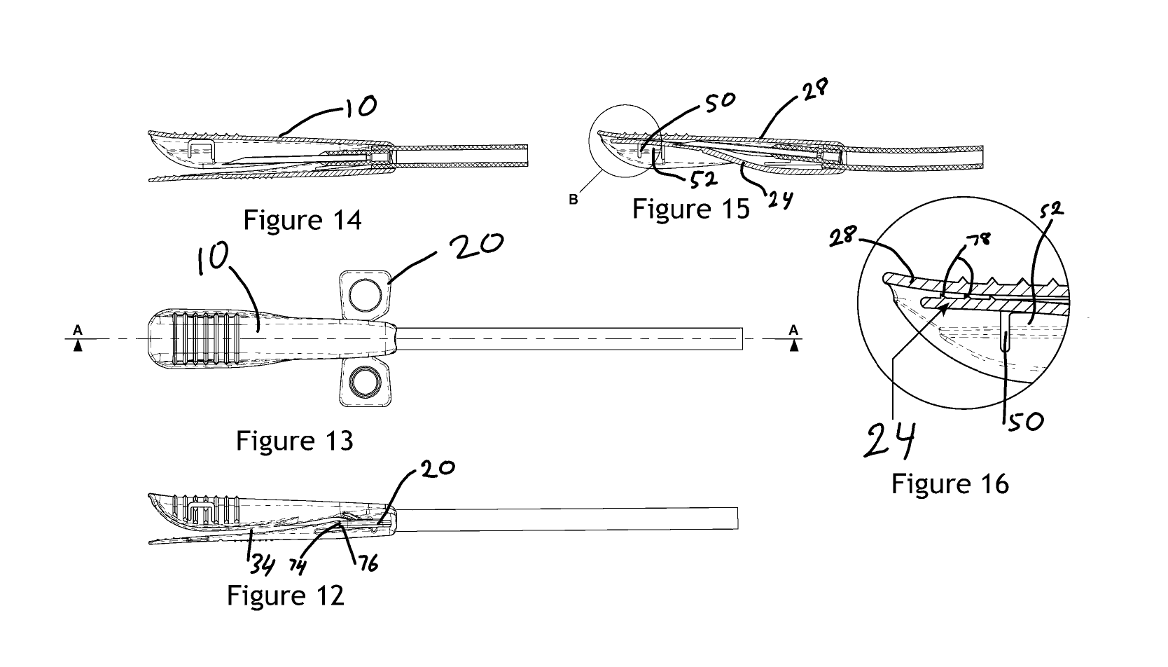

Referring to FIGS. 1 to 16 there is shown a needle guard 10 according to a first embodiment of the invention. The needle guard is adapted to be mounted on mounted on a needle assembly 12, as shown in FIG. 9. The needle assembly 12 comprises a conduit 14 and a needle 16 mounted on a needle block 18 with the conduit connected to the needle block 18. The needle block 18 has two flexible wings 20 that extend on either side of the block to enable a used to more easily grasp and manipulate the needle assembly 12.

The needle guard 10 comprises concave portion 22 and tongue portion 24. Portions 22 and 24 joined together at the rear 26 of the guard.

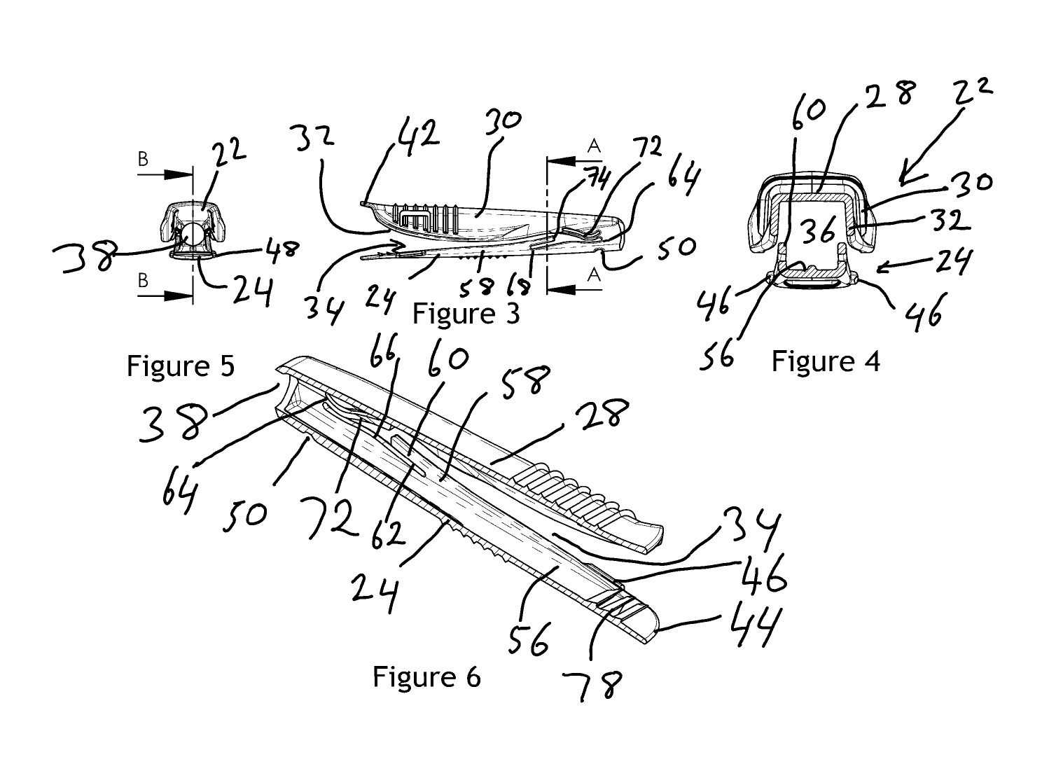

As best seen in FIG. 4, in this embodiment the concave portion 22 is U-shaped and comprises upper wall 28 with two opposed side walls 30. The tongue 24 is sized to fit within the two side walls 30. The tongue 24 is adapted to move relative to upper portion 22 between open and closed positions.

However, the concave portion 22 need not have definite top and side walls and may have a continuous curve in end view, such as a semicircular or other cross section.

In the “open” position the tongue 24 lies below the lower edges 32 of side walls 30 and as seen in FIG. 3, there is a longitudinally extending slot, opening or space 34 between the tongue 24 and the side walls 30.

The upper portion 22 and tongue 24 thus define a longitudinally extending passageway 36 with a front aperture 40 at the front end 42 of the guard. There is a rear aperture 38 in the guard's rear end 26. The guard 10 is adapted to be mounted onto the conduit 14 with the conduit 14 passing through rear aperture 38, along passageway 36 and out of front aperture 40 with the needle 16 and needle block 18 outside of the guard 10 forward of forward end 42, as seen in FIGS. 9 to 11.

The guard 10 may be moved along the conduit so as to draw the needle 16 and needle block 18 into the guard 10.

The lower edges 32 are shaped so as to curve away from the tongue 24 toward the front end 42 and so the slot 34 has a large size at the front end 42 and progressively narrows toward the rear end 26. This is not critical but aids a user in drawing the needle assembly 14 into the guard 10.

The upper extent (or upper side) of slot 34 is defined by the lower edges of side walls 30. The lower extent (or lower side) is defined by the tongue 24. In the open position the base wall 56 of tongue 24 is generally parallel with upper wall 28. Side walls 58 extend upwards from base wall 56. As best seen in FIG. 3, theses side walls 58 become taller toward the rear of the guard. The lower edges 32 of side walls 30 also rise upwards and so the slot 34 is slopped upwards relative to the longitudinal direction in the rearwards direction.

Toward the rear of the guard the side walls 58 cease to be connected to the base wall 56 and arms 60 extend rearwards, each defining a slot 62 between the arms 60 and base wall 56.

Beyond the rearward extent of the arm 60 the lower edge 32 curves downwards and joins the tongue 24 to define the rearward extent 64 of the slot 34. The lower surface 66 between the front 68 of slot 62 and rear end 64 of slot 34 may be formed with generally flat or may be formed slightly convex. The rear end 64 of the slot 34 may be a simple rounded end or may be formed with an oversized portion 70, as best seen in FIG. 9.

At least one guide wall 72 is provided and is positioned to urge the conduit 14 and/or needle block 18 downwards toward the base wall 56 of tongue 24. The guide wall 72 may extend sideways from one or both side walls, as in this embodiment, or may extend internally in the passageway from upper wall 28. The guide wall 72 holds the needle block and wings tightly and so improves the feeling of the needle assembly being securely and safely held. Each guide wall 72 is defined by a slot 73 and so may flex upwards to increase the size of the slot 34.

After use the needle is withdrawn into the guard 10 by moment of the guard 10 relative to the conduit 14, usually by sliding the guard 10 along the conduit 14. The guard moves toward the needle and the wings engage the upper or lower edges of slot 34 and aid in guiding the needle block into the guard 10. The guard 10 continues its forward movement and the wings 20 move rearwards along slot whilst the needle 16 and needle block 18 move into the passageway 36.

Because the slot 34 is “open” when the guard 10 is in the open position drawing the needle assembly into the guard does not require any increase in separation of the tongue 24 and concave portion 22. As such there is relatively little force required to withdraw the needle assembly.

The arms 60 are primarily provided so that as the wings pass and start to change direction as the slot 34 curves downwards the arms 60 deflect downwards and so that there is less resistance (due to deflection of the wings) than otherwise. If desired the slot 62 may be omitted so that the side walls 58 are continuous.

The block 18 is moved backwards until the rear edges of the wings 20 reach the rear ends 64 of slots 34 and/or clear the free ends of arms 60.

The distance between the rear 64 of the slot 34 and the rear 74 of the arms 64 is preferably the same or more than the length of the wings 20

The needle assembly is drawn backward until the front edges 76 of the wings 20 clear the rear end 74 of arms 64 and the front edges 76 just sit behind the rear 74 of the arms 64. However the distance may be less and this may require the wings to be elastically distorted by generally “waviness” or by curving the rear end portion into the oversize rear portion of slot 34.

If desired the slot 62 may be sized so that the wings 20 can be slid forward into the slots 62 to act as an additional retention mechanism, but this is not essential. Once the wings have cleared the rear end 74 they may be moved forwards to move the front portion of the wings into slot 62. The slots 62 may preferably be configured so that forward movement of the needle assembly 12 will result in the wings 20 entering the slots 62 without the user needing to actively guide them.

The passageway 36 remains open and potentially a person could insert a body part, such as a small finger, into the open end of the guard and impale themselves on the needle 16. In addition, a person may deliberately draw the needle block backwards and manipulate the needle block to remove the wings from slot 62 and move the needle out of the guard. Accordingly the guard has a closure mechanism for the passageway, which in the preferred embodiment utilizes the tongue 24.

The lower tongue 24 may be rotated about a general hinge line 50 and/or generally flexed relative to concave portion 22 to a locked position in which all or part of the tongue lies between opposed side walls 30, as seen in FIGS. 8 and 15. The front end 44 of the tongue 24 is sized so that it may be moved upwards to lie against or near to the upper wall 28, as seen in FIGS. 8 and 15.

Toward the front end 44 of tongue 24 are two sideways extending wings or protrusions 46. The separation of the outer edges 48 of protrusions 46 is slightly greater than the separation of the side walls when the front end 44 of tongue 24 is against or near to the upper wall 28. Each side wall 28 has an opening 50 into which the protrusions may extend when in the closed position.

As best seen in FIG. 1 the lower edges of the side walls 30 are preferably flared sideways at 54 so as to aid initial entry of the protrusions 46 between the side walls.

The guard is preferably formed of flexible plastics material and so the side walls 30 may flex outwards to accommodate the protrusions 46 as the tongue is moved to the closed position, with the side walls snapping back to their un-deflected state when the protrusions align with and enter the openings 50. The snap action provides an audible click that reassures the user that the tongue 24 is securely locked in the closed position.

In the embodiment shown each opening 50 is generally U-shaped and so defines a leg 52 that may flex, rather than relying only on flexing of the side walls 30 as a whole. However, if desired, the shape of the opening 50 may merely correspond to the shape of the protrusions, such as a rounded rectangle.

Thus once the tongue 24 has been moved to the closed position the needle assembly is substantially irreversibly locked within the guard and cannot be accidentally withdrawn.

A used needle typically has some fluids on itself and it is preferable that any such fluids remain within the guard. Accordingly, the side edges of the tongue 20 and side walls 30 may be formed to be substantially fluid tight. This may be by having an interference fit sideways between the walls 30 and tongue 20 or by having any gaps between themselves small enough to resist fluid flow.

The upper surface of the tongue 20 is preferably formed with a series of flexible sealing fins 78 located toward the front of the tongue. In the closed position the sealing fins 78 engage against the underside of upper wall 28 and substantially seal the front end of the passageway. The tongue and upper wall 28 are preferably formed so that when locked in position the tongue urges the fins against the upper wall 28. In the preferred embodiment the fins are located on the open side of the locking protrusions 48. If desired the fins may be located in the region of the locking protrusions 48 or rearwards of them.

As best seen by comparing FIGS. 14 and 15, the action of moving the tongue to the locked position also moves the free end of the needle upwards. This rotary movement is accommodated at the rear of the guard by the guide wall 72 flexing upwards whilst maintaining pressure on the needle block. The wings 20 remain engaged within the slots 62 and so bend relative to the needle block 18.

As best seen in FIG. 8 when the tongue has been moved to the closed position the slot 34 is substantially closed and any forward directed force on the needle assembly 12 cannot move the needle assembly forward. In particular the end 74 of arms 60 (or wall 58 if slot 62 is not present) is adjacent or overlies the lower edge 32 of side wall 30.

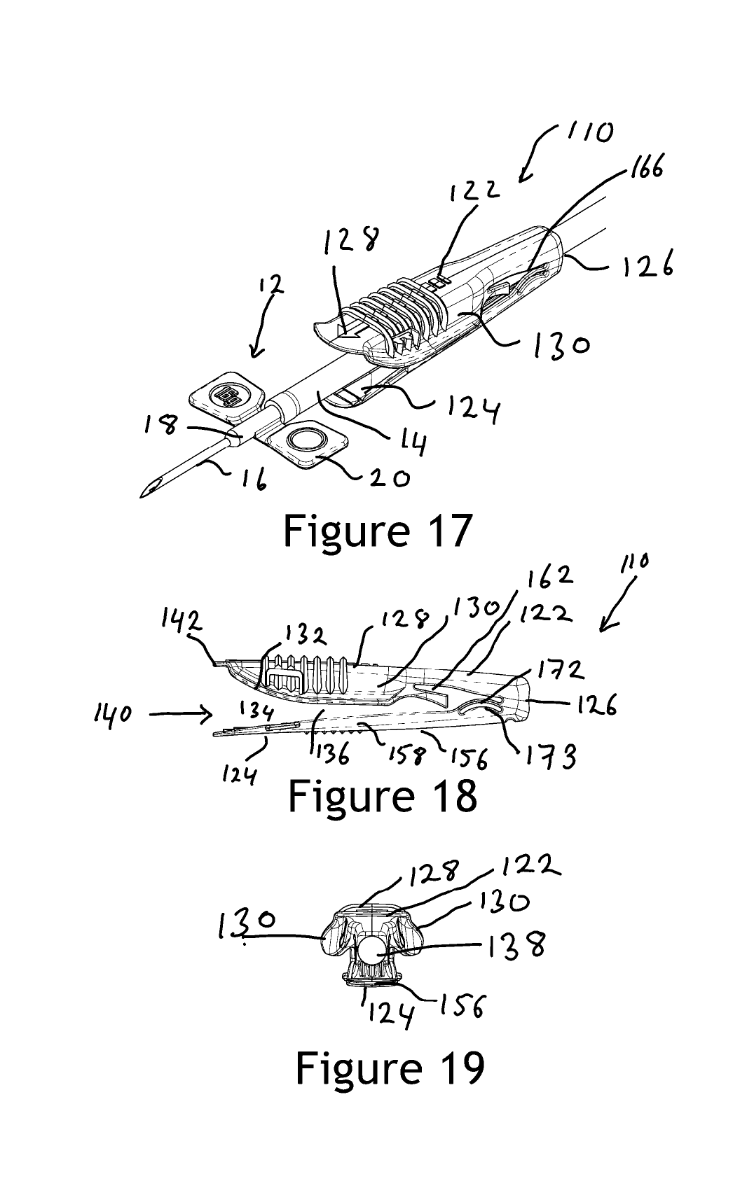

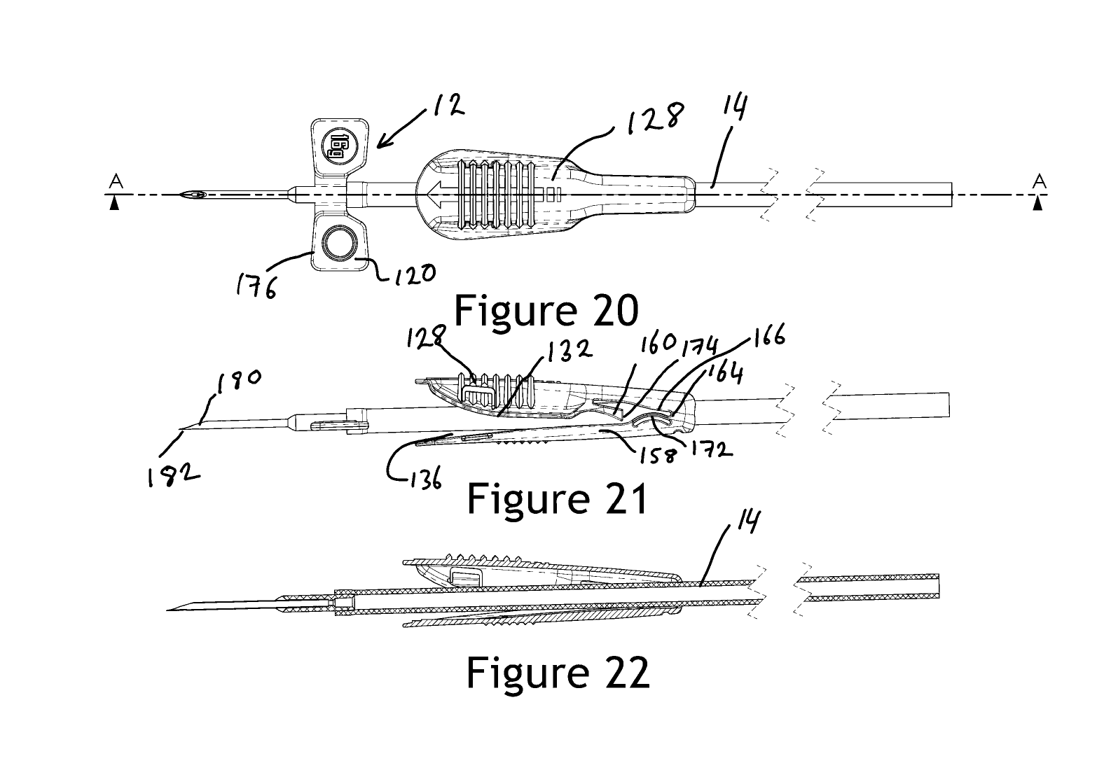

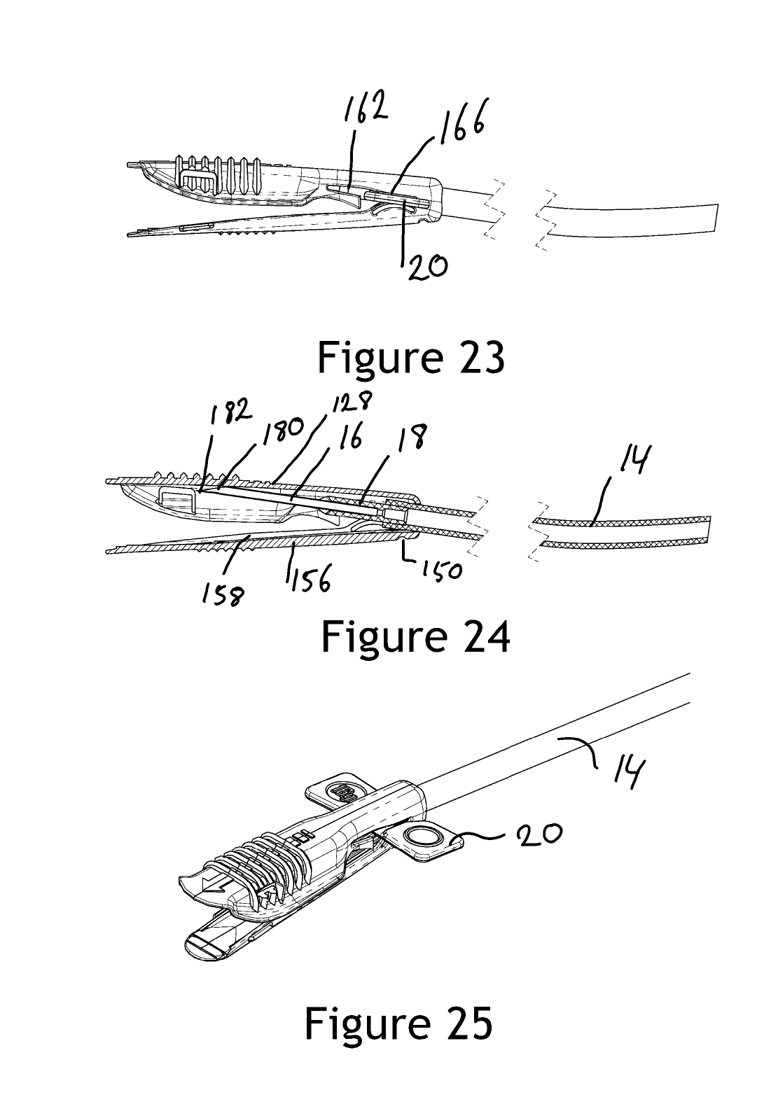

Referring to FIGS. 17 to 28 there is shown a needle guard 110 according to a second embodiment of the invention.

The needle guard is adapted to be mounted on mounted on a needle assembly 12, as shown in FIG. 19. The needle assembly 12 comprises a conduit 14 and a needle 16 mounted on a needle block 18 with the conduit connected to the needle block 18. The needle block 18 has two flexible wings 20 that extend on either side of the block to enable a used to more easily grasp and manipulate the needle assembly 12.

The needle guard 110 is similar to the needle guard 10 of the first embodiment but the arrangement for locking the needle assembly 12 is slightly different. However, for completeness the needle guard 110 will be described fully.

The needle guard 110 comprises concave portion 122 and tongue portion 124. Portions 122 and 124 joined together at the rear 126 of the guard.

As best seen in FIG. 19, in this embodiment the concave portion 122 is U-shaped and comprises upper wall 128 with two opposed side walls 130. The tongue 124 is sized to fit within the two side walls 130. The tongue 124 is adapted to move relative to upper portion 122 between open and closed positions.

However, the concave portion 122 need not have definite top and side walls and may have a continuous curve in end view, such as a semicircular or other cross section.

In the “open” position the tongue 124 lies below the lower edges 132 of side walls 130 and as seen in FIG. 18, there is a longitudinally extending slot, opening or space 134 between the tongue 124 and the side walls 130.

The concave portion 122 and tongue 124 thus define a longitudinally extending passageway 136 with a front aperture 140 at the front end 142 of the guard. There is a rear aperture 138 in the guard's rear end 126. The guard 110 is adapted to be mounted onto the conduit 14 with the conduit 14 passing through rear aperture 138, along passageway 136 and out of front aperture 140 with the needle 16 and needle block 18 outside of the guard 110 forward of forward end 142, as seen in FIG. 1.

The guard 110 may be moved along the conduit so as to draw the needle 16 and needle block 18 into the guard 110.

The lower edges 132 of side walls 130 are shaped so as to curve away from the tongue 124 toward the front end 142 and so the slot 134 has a large size at the front end 142 and progressively narrows toward the rear end 126. This is not critical but aids a user in drawing the needle assembly 14 into the guard 110.

The upper extent (or upper side) of slot 134 is defined by the lower edges 132 of side walls 130. The lower extent (or lower side) is defined by the tongue 124. In the open position the base wall 156 of tongue 124 is angled slightly downwards relative to upper wall 128 but this is not critical. Side walls 158 extend upwards from base wall 156 and provide stiffening to the tongue 124. They also serve to keep the needle assembly and conduit within the needle guard but may be replaced by other stiffening structures, such as ribs on the underside of the base wall.

Toward the rear of the guard a curved flexible arm 172 extends upwards away from the base 156 on each side of the tongue 124. A slot 173 allows the flexible arm 172 to be flexed toward the base 156. The flexible arms 172 serve a similar function as curved portions 72 in the first embodiment. There are two flexible arms 172 and each is joined to the tongue 124 at both ends. However, the flexible arm 172 may be free at its rearward end. The same applies to the guide walls 72 of the first embodiment.

On each of the side walls 130 of the channel member an arm 160 extends toward the tongue member 124, with the free end 174 of the arms 160 located forward of flexible arms 172 and close to the tongue 124. As best seen in FIG. 18, in side view, the gap between the ends of arms 160 is less than the thickness of the wings 20 of needle assembly 12. Beyond the rearward extent of the arm 160 the upper edge extends downwards and joins the tongue 124 to define the rearward extent 164 of the slot 134. The portion 166 of upper edge that overlies the flexible arm 172 is preferably generally planar and angled toward the upper wall in the forward direction. The gap between this portion 166 and the flexible arm 172 is preferably less than the thickness of the wings of the needle guard.

Instead of extending at the sides of the passageway 136 one or more flexible arms 172 may extend internally in the passageway 136 from lower wall 156. The flexible arms 172 holds the needle block and wings tightly and so improves the feeling of the needle assembly being securely and safely held.

After use the needle is withdrawn into the guard 110 by moment of the guard 110 relative to the conduit 14, usually by sliding the guard 110 along the conduit 14. The guard moves toward the needle and the wings engage the upper or lower edges of slot 134 and aid in guiding the needle block into the guard 110. The guard 110 continues its forward movement and the wings 120 move rearwards along slot whilst the needle 16 and needle block 18 move into the passageway 136.

Because the slot 134 is “open” when the guard 110 is in the open position drawing the needle assembly into the guard does not require any increase in separation of the tongue 124 and concave portion 122. As such there is relatively little force required to draw the needle assembly into the guard.

As the wings pass the arms 160, the arms 160 deflect upwards and the rear edge of the wings then engage the flexible arm 172 and deflect the flexible arm portions 172 downwards.

The block 18 is moved backwards until the rear edges of the wings 120 reach the rear ends 164 of slots 134 and/or the front edges 176 clear the free ends of arms 160.

The distance between the rear 164 of the slot 134 and the rear 174 of the arms 164 is preferably the same or more than the length of the wings 120.

The needle assembly is drawn backward until the front edges 176 of the wings 120 clear the rear end 174 of arms 164 and the front edges 176 just sit behind the rear 174 of the arms 164. However the distance may be less and this may require the wings to be elastically distorted by generally “waviness” or by curving the rear end portion into the oversize rear portion of slot 134.

Where the wings easily clear the rear end 174 of arms 164, as they do so, the arms 160 move back to their un-deflected state, reducing the size of the slot 134 to less than the thickness of the wings. Thus without any additional measures, the needle assembly is prevented from moving toward the open end. Simultaneously, the flexible arm portions 172 rotate the front of the needle assembly upwards so the wings are held against portion 166 and the needle 16 extends generally parallel to the portion 166. These two actions tend to generate a reassuring clicking sound that indicates the wings 120 have passed the arms 164.

As seen in FIG. 18, the free end 174 of each arm 164 extends generally perpendicular to the direction of travel of the needle assembly 12 and forward movement of the needle assembly will be prevented by engagement on the free end 174. Such engagement will not generate any significant upward deflection of the arms. As mentioned, the gap between the free ends 174 of the arms 164 is less than the thickness of the wings 20 and so even if a user deliberately manipulates the needle assembly within the guard, the arms 164 prevent forward motion unless deliberately flexed upwards.

If desired the slot 162 may be sized so that the wings 120 can be slid forward into the slots 162 to act as an additional retention mechanism, but this is not essential.

The passageway 136 remains open and potentially a person could insert a body part, such as a small finger, into the open end of the guard. The arrangement in this embodiment with the angled upper wall portion 166 and flexible arms 172 urging the needle assembly upwards against this wall portion means that the cannula is angled toward the upper wall 128, as best seen in FIG. 24. In preferred configurations of this embodiment the cannula 16 is angled so that it is urged against the upper wall 128. As seen in FIG. 24, when the bevel 180 of the cannula is upwards, the free end 182 of the cannula is still very close to the upper wall. When the bevel 180 of the cannula is downwards, the free end 182 of the cannula will bear against the upper wall 128. As such the risk that a user will impale themselves on the needle 16 is reduced compared to the first embodiment.

As with the first embodiment, the guard has a closure mechanism for the passageway, which in the preferred embodiment utilizes the tongue 124.

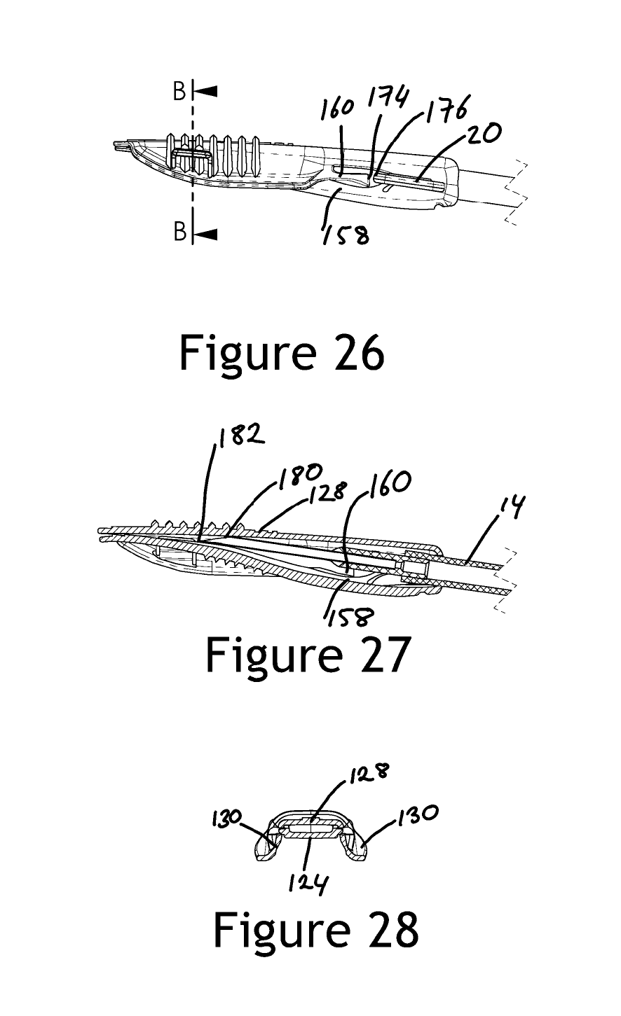

The lower tongue 124 may be rotated about a general hinge line 150 and/or generally flexed relative to concave portion 122 to a locked position in which all or part of the tongue lies between opposed side walls 130, as seen in FIGS. 26 to 28. The front end 144 of the tongue 124 is sized so that it may be moved upwards to lie against or near to the upper wall 128, as seen in FIGS. 18 and 15.

Toward the front end 144 of tongue 124 are two sideways extending wings or protrusions 146. The separation of the outer edges 148 of protrusions 146 is slightly greater than the separation of the side walls when the front end 144 of tongue 124 is against or near to the upper wall 128. Each side wall 128 has an opening 150 into which the protrusions may extend when in the closed position.

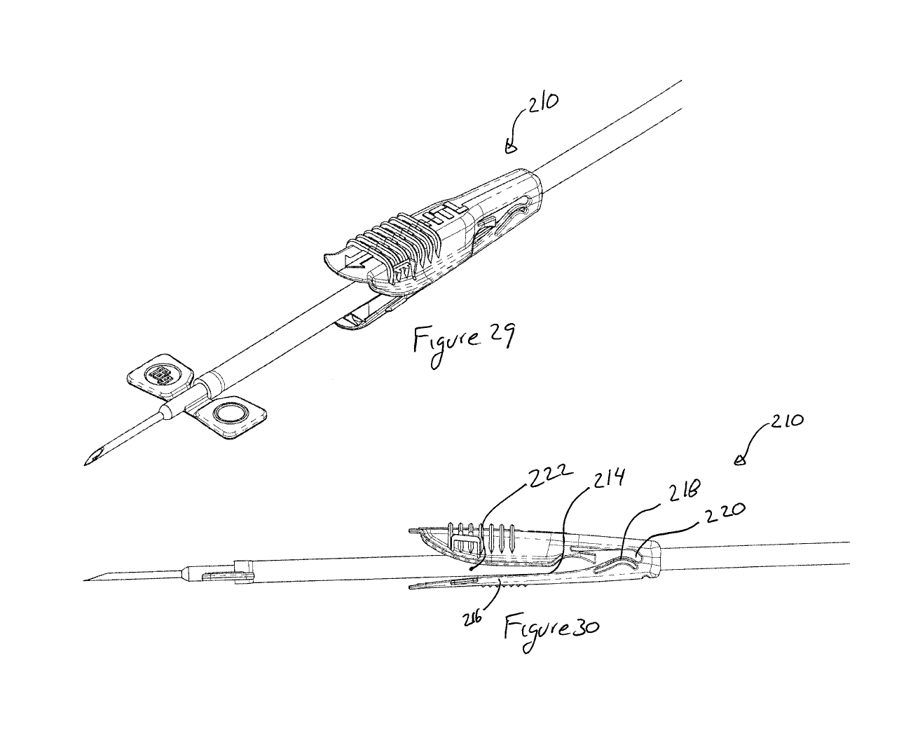

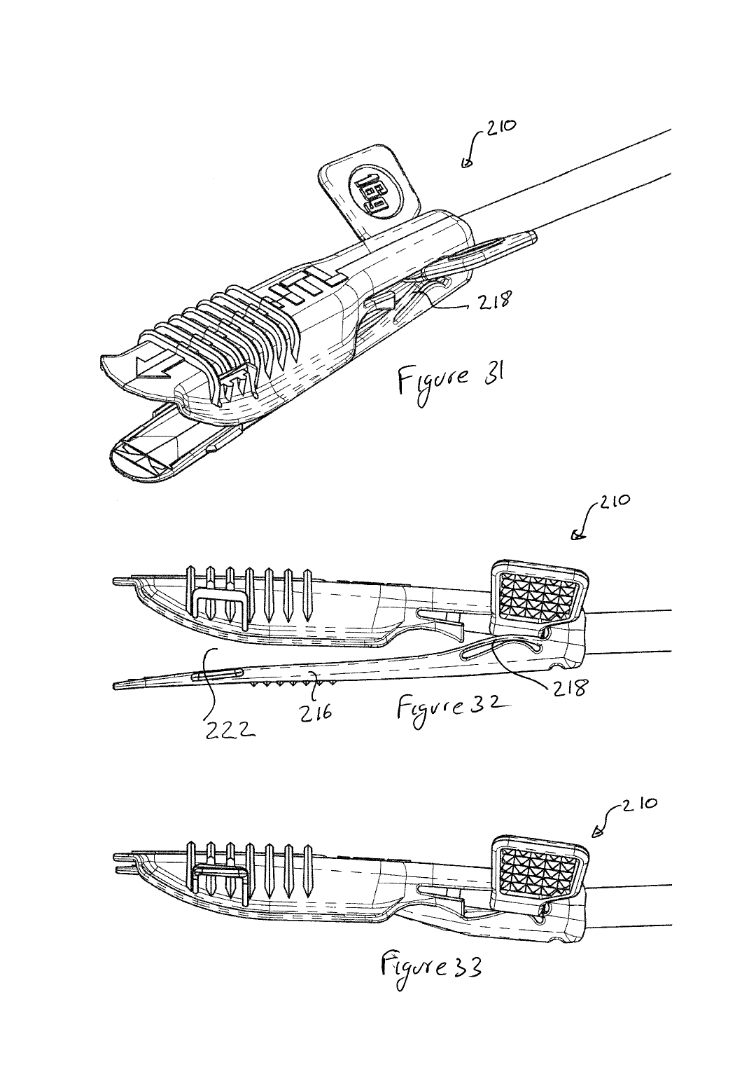

As best seen in FIGS. 19 and 28 the lower edges of the side walls 130 are preferably flared sideways at 154 so as to aid initial entry of the protrusions 146 between the side walls.

The guard is preferably formed of flexible plastics material and so the side walls 130 may flex outwards to accommodate the protrusions 146 as the tongue is moved to the closed position, with the side walls snapping back to their un-deflected state when the protrusions align with and enter the openings 150. The snap action provides an audible click that reassures the user that the tongue 124 is securely locked in the closed position.

In the embodiment shown each opening 150 is generally U-shaped and so defines a leg 152 that may flex, rather than relying only on flexing of the side walls 130 as a whole. However, if desired, the shape of the opening 150 may merely correspond to the shape of the protrusions, such as a rounded rectangle.

Thus once the tongue 124 has been moved to the closed position the needle assembly is substantially irreversibly locked within the guard and cannot be accidentally withdrawn.

A used needle typically has some fluids on itself and it is preferable that any such fluids remain within the guard. Accordingly, the side edges of the tongue 120 and side walls 130 may be formed to be substantially fluid tight. This may be by having an interference fit sideways between the walls 130 and tongue 120 or by having any gaps between themselves small enough to resist fluid flow.

The upper surface of the tongue 120 is preferably formed with a series of flexible sealing fins 178 located toward the front of the tongue. In the closed position the sealing fins 178 engage against the underside of upper wall 128 and substantially seal the front end of the passageway. The tongue and upper wall 128 are preferably formed so that when locked in position the tongue urges the fins against the upper wall 128. In the preferred embodiment the fins are located on the open side of the locking protrusions 148. If desired the fins may be located in the region of the locking protrusions 148 or rearwards of them.

As best seen in FIGS. 26 and 27 when the tongue has been moved to the closed position the slot 134 is closed, with the arms 164 engaging the side walls 158 and deflected upwards. Any forward directed force on the needle assembly 12 cannot move the needle assembly forward.

Although the preferred embodiments utilize generally planar upper, lower and side walls 28, 24 and 30 and 128, 124 and 130 respectively it will be appreciated that the concave portions 22 and 122 may be a channel or the like without specific top and side walls having an opening that receives the lower member 24 and 124. As an example a channel having a semicircular cross section would suffice. It will also be appreciated that the use of “upper” and “lower” is also for convenience and the orientation of the various components is not critical.

Whilst the preferred embodiments described have a generally planar tongue members 24 and 124 that is received within the respective concave member 22 or 122 along substantially its entire length, closure of the slot(s) may be by the tongue member having a wider portion that bears against the lower side edges of the concave members 22 or 122. The tongue members may have a narrower portion that extends into the concave portions 22 or 122 and closes the passageway.

FIGS. 29 to 30 show a needle guard 210 according to another embodiment mounted on a needle assembly 212. The needle guard 210 is substantially the same as the needle guard 110 of FIGS. 17 to 28. The needle guard 210 differs from needle guard 110 in that the transition from the upper surface 214 of base 216 to the curved flexible arm 218 is smoother. The rearward end portion 220 of the slot 222 is also a different shape. This aids compatibility when a winged needle is used upside-down.

Unless the context clearly requires otherwise, throughout the description and any claims the words “comprise”, “comprising”, and the like are to be construed in an inclusive sense as opposed to an exclusive or exhaustive sense; that is to say, in the sense of “including, but not limited to”.

The features of the invention described or mentioned in this document may be combined in any combination of features where features are not mutually exclusive.

It will be apparent to those skilled in the art that many obvious modifications and variations may be made to the embodiments described herein without departing from the spirit or scope of the invention.

1. A needle guard for use with a needle assembly having a needle mounted on a needle block, the guard comprising:
an elongate concave channel member having a longitudinally extending opening between two longitudinally extending side edges, the concave channel member defining at least part of a passageway having a forward end, for receiving at least part of the needle assembly, and a rear end;
an elongate tongue member, in an open position, extending longitudinally adjacent the opening and defining at least one slot between itself and at least one of the side edges, the at least one slot having an open forward end adapted to receive at least one wing of a winged needle block, and a rear end;
at least a portion of the tongue member movable to a closed position to lie within the channel member to close, block or substantially close or block access to the passageway.

2. (canceled)

3. The needle guard of claim 1 comprising at least one first flexible arm including a rearward facing surface configured to resist forward motion of an object located in the slot rearwards of the rearward facing surface.

4. The needle guard of claim 3 wherein at least one first flexible arm, in the open position, extends into or overlaps the slot between its ends to reduce the effective height of the slot.

5. The needle guard of claim 4 wherein at least one flexible arm, in the closed position, extends into or overlaps the slot between its ends to close the slot.

6. The needle guard of claim 1 wherein the tongue includes sealing structure adapted to engage the channel member in the closed position and substantially seal the front end of the passageway against leakage of liquid from the passageway to the environment via the front end.

7. (canceled)

8. The needle guard of claim 6 wherein the sealing structure comprises flexible sealing fins and, in the closed position, the sealing fins substantially seal the front end of the passageway.

9. The needle guard of claim 8 wherein the flexible sealing fins are located on an upper surface of the tongue and, in the closed position, the sealing fins engage against an underside of the channel member.

10. (canceled)

11. The needle guard of claim 1 comprising at least one guide portion adapted to bias the needle assembly, when in the retracted position, toward the tongue or the channel member.

12. The needle guard of claim 11 wherein, the at least one guide portion is adapted to bias the needle assembly, when in the retracted position, toward the tongue or the channel member so that a portion of the cannula at or near the free end of the cannula is close to or engages the surface of the respective tongue or the channel member.

13. The needle guard of claim 12 wherein at least one of the at least one guide portion comprises at least one biasing member.

14. The needle guard of claim 13 wherein at least one biasing member comprises at least one second flexible arm.

15. The needle guard of claim 14 wherein the at least one second flexible arm defines part of the slot and wherein, in the open position, the at least one slot communicates with at least one wing-receiving recess, for receiving at least one wing of a winged needle block.

16. (canceled)

17. The needle guard of claim 1 wherein, in the open position, when viewed side on, there is gap between the at least one side edge and the tongue member.

18. (canceled)

19. The needle guard of claim 20 wherein the concave channel member comprises a top wall and two opposed first side walls.

20. The needle guard of claim 1 wherein, in the closed position, side edges of the tongue member and the concave channel member form a substantially fluid tight seal.

21. The needle guard of claim 19 wherein the tongue member comprises a lower wall and two opposed second side walls.

22. The needle guard of claim 20 wherein, in the closed position, any gaps between the tongue member and the concave channel member are small enough to resist fluid flow.

23. The needle guard of claim 20 wherein, in the closed position, side edges of the tongue member and the concave channel member are interference fit to resist fluid flow.

24. (canceled)

25. (canceled)

26. The needle guard of claim 1 wherein, in the closed position, the tongue member bears against the side edges to close the slot.

27. (canceled)

28. (canceled)
